(19) United States
(12) Patent Application Publication
(10) Pub. No.: US 2020/0196987 A1
(43) Pub. Date: Jun. 25, 2020

(54) METHOD AND SYSTEM TO MANAGE BEAMFORMING PARAMETERS BASED ON TISSUE DENSITY

(71) Applicant: General Electric Company, Schenectady, NY (US)

(72) Inventor: Jeong Seok Kim, Seongnam-SI (KR)

(21) Appl. No.: 16/226,783

(22) Filed: Dec. 20, 2018

(51) Int. Cl. A61B 8/08; A61B 8/00

(52) U.S. Cl. CPC A61B 8/5207; A61B 8/469; A61B 8/14; A61B 8/5223; A61B 8/5246; A61B 8/4483

(57) ABSTRACT

An ultrasound system and method are provided. The system comprises a probe that is operable to transmit ultrasound signals and receive echo ultrasound signals from a region of interest (ROI) and a processing circuitry. The processing circuitry performs a first beamforming operation on at least a portion of the echo ultrasound signals to generate a first ultrasound dataset, corresponding to at least a portion of a first ultrasound image. The first beamforming operation performs beamforming for a subregion of the ROI utilizing an initial time delay as a beamforming parameter. The system applies a deep learning network (DLN) model to a local region of the first ultrasound dataset to identify at least one of a tissue type or density characteristic associated with the local region. The system adjusts the beamforming parameter to use a density adjusted (DA) time delay based on the at least one of a tissue type or density characteristic of the local region, to form a density adjusted beamforming (DAB) parameter and performs a second beamforming operation on at least a portion of the echo ultrasound signals, based on the DA time delay for the DAB parameter, to generate a second ultrasound dataset.

### FIELD

Aspects of the present disclosure relate to medical imaging. More specifically, certain embodiments relate to methods and systems for managing beamforming parameters based on automatic labeling of tissue type and/or density in ultrasound imaging.

### BACKGROUND OF THE INVENTION

Various medical diagnostic imaging techniques may be utilized in connection with imaging organs, bone and soft tissue in a human body. Ultrasound imaging uses real time, non-invasive high frequency sound waves to produce ultrasound images of anatomical structures such as organs, tissue, vessels, and objects inside the human body. During ultrasound imaging, ultrasound datasets (including, e.g., volumetric imaging datasets during 3D/4D imaging) are acquired and utilized to generate and render corresponding images (e.g., via a display) in real-time or post acquisition. Ultrasound images produced or generated during medical imaging may be presented as two-dimensional (2D), three-dimensional (3D), and/or four-dimensional (4D) images (essentially real-time/continuous 3D images).

Conventional ultrasound systems and methods experience certain limits. Conventional ultrasound systems perform beamforming utilizing beamforming parameters that are based on an assumption that ultrasound signals travel at a constant predetermined velocity through all types of anatomy. Thus it is assumed the ultrasound and signals have a constant predetermined propagation time from a focal point within a region of interest to an individual transducer element within an ultrasound probe. However, in reality, the ultrasound signals travel at different velocities through different types of anatomy, based on the tissue type and density of the anatomy. Consequently, conventional ultrasound systems fail to account, within the beamforming process, for the different types of tissue in the areas being images, resulting in imaging operations that can be inefficient and/or ineffective, and potentially unduly costly when scans must be repeated. In general, despite the difference in the internal structure of the human body, conventional systems calculate the beamforming parameter time delays based on a predetermined velocity that is assumed for all tissue (e.g., about 1540 m/s).

Further, different patients exhibit differences in the density of the tissue, even within common anatomies between different patients. For example, two patients may exhibit differences between a degree of hardness or fat within a particular organ (e.g., one patient has a hard liver, while another patient has a fatty liver). When convention systems perform beamforming, using time delays that are based on an assumed ultrasound velocity, the conventional systems form ultrasound images that have a resolution that does not account for fluctuations in the tissue characteristics of individual patients.

Additional limitations and disadvantages of conventional and traditional approaches will become apparent to one of skill in the art, through comparison of such systems with some aspects of the present disclosure, as set forth in the remainder of the present application with reference to the drawings.

### BRIEF DESCRIPTION

In accordance with embodiments herein, an ultrasound system is provided. The system comprises a probe that is operable to transmit ultrasound signals and receive echo ultrasound signals from a region of interest (ROI) and a processing circuitry. The processing circuitry performs a first beamforming operation on at least a portion of the echo ultrasound signals to generate a first ultrasound dataset, corresponding to at least a portion of a first ultrasound image. The first beamforming operation performs beamforming for a subregion of the ROI utilizing an initial time delay as a beamforming parameter. The system applies a deep learning network (DLN) model to a local region of the first ultrasound dataset to identify at least one of a tissue type or density characteristic associated with the local region. The system adjusts the beamforming parameter to use a density adjusted (DA) time delay based on the at least one of a tissue type or density characteristic of the local region, to form a density adjusted beamforming (DAB) parameter and performs a second beamforming operation on at least a portion of the echo ultrasound signals, based on the DA time delay for the DAB parameter, to generate a second ultrasound dataset.

Optionally, the processing circuitry may be further operable to segment the first ultrasound dataset into multiple local regions and, for at least a portion of the local regions, repeat the first and second beamforming operations, applying the DLN model and adjusting the DAB parameter. The TDB parameter and DAB parameter may include different first and second sets of time delays that may be utilized during the first and second beamforming, respectively, in connection with a common segment of the ROI. The first and second beamforming operations may be performed on a common portion of the echo ultrasound signals. The probe may be operable to perform first and second scans of the ROI, during which first and second sets of the echo ultrasound signals may be received. The first scan may be performed before the first beamforming operation. The second scan may be performed after the first beamforming operation and before the second beamforming operation.

Optionally, he DLN model may classify the local regions to correspond to one of at least two different types of tissue, the types of tissue including at least two of air, lung, fat, water, brain, kidney, liver, myocardium, or bone. The TDB parameter may include a first time delay value associated with a reference density. The processing circuitry may be operable to adjust the TDB parameter to form the DAB parameter by changing the first time delay value to a second time delay value associated with a predicted density corresponding to the at least one of a tissue type or density characteristics identified by the DLN model.

Optionally, the second time delay value may be determined based on a propagation time from an array element of the probe to a focal point in the ROI utilizing a predicted speed of sound that may be determined based on the at least one of a tissue type or density characteristics identified by the DLN model. The second ultrasound dataset may be based on second ultrasound signals that are received after adjusting the beamforming parameter. The second ultrasound dataset may correspond to a second ultrasound image. The processing circuitry may be operable to segment the first ultrasound dataset into a two-dimensional array of the local regions, wherein each of the local regions may correspond to a different portion of the ultrasound image.

In accordance with embodiments herein, a computer implemented method is provided. The method utilizes an ultrasound probe to transmit ultrasound signals and receive echo ultrasound signals from a region of interest. The method is under control of processing circuitry. The method performs first beamforming on at least a portion of the echo ultrasound signals to generate a first ultrasound dataset, corresponding to a first ultrasound image, based on a time delay beamforming (TDB) parameter and applies a deep learning network (DLN) model to the local regions to identify at least one of a tissue type or density characteristics associated with corresponding portions of the ROI in the associated local regions. The method adjusts the TDB parameter, based on the at least one of a tissue type or density characteristics of the corresponding local regions, to form a density adjusted beamforming (DAB) parameter and performs second beamforming on at least a portion of the echo ultrasound signals, based on the DAB parameter, to generate a second ultrasound dataset.

Optionally, the first and second beamforming may be performed on a common portion of the echo ultrasound signals. The probe may be operable to perform first and second scans of the ROI, during which first and second sets of the echo ultrasound signals are received. The first scan may be performed before the first beamforming operation. The second scan may be performed after the first beamforming operation and before the second beamforming operation. The DLN model may classify the local regions to correspond to one of at least two different types of tissue, the types of tissue including at least two of air, lung, fat, water, brain, kidney, liver, myocardium, or bone.

Optionally, the TDB parameter may include a first time delay value associated with a reference density. The processing circuitry may be operable to adjust the TDB parameter to form the DAB parameter by changing the first time delay value to a second time delay value associated with a predicted density corresponding to the at least one of a tissue type or density characteristics identified by the DLN model. The second time delay value may be determined based on a propagation time from an array element of the probe to a focal point in the ROI utilizing a predicted speed of sound that may be determined based on the at least one of a tissue type or density characteristics identified by the DLN model. The second ultrasound dataset may be based on second ultrasound signals that may be received after adjusting the DAB parameter. The second ultrasound dataset may correspond to a second ultrasound image.

In accordance with embodiments herein, a system is provided. The system comprises memory to store program instructions and one or more processors. When executing the program instructions, the processors obtain a collection of reference images for a patient population, the reference images representing ultrasound images that are obtained from a patient population having different types of tissue for one or more anatomical regions and analyze the collection of reference images utilizing a deep learning network (DLN) to define a DLN model that is configured to identify different types of anatomical regions and different density properties within the corresponding anatomical regions.

Optionally, the one or more processors may be configured to analyze the collection of reference images by performing one or more convolutions and up sampling operations to generate a feature map. The one or more processors may be configured to train the DLN model by minimizing a sigmoid cross loss objective.

### BRIEF DESCRIPTION OF THE DRAWINGS

FIG. 1 illustrates a process for managing beamforming parameters based on tissue characteristics in accordance with embodiments herein.

FIG. 2A illustrates a graphical representation of a process in which the DLN model is built in accordance with embodiments herein.

FIG. 2B illustrates an alternative graphical representation of a process in which the DLN model is built in accordance with an embodiment herein.

FIG. 3 illustrates a process for managing beamforming parameters based on tissue characteristics in accordance with embodiments herein.

FIG. 4 illustrates a block diagram of an implementation that applies a DLN model in accordance with an embodiment herein.

FIG. 5 illustrates a density table designating different tissue types, along with corresponding densities, velocities, impedances and attenuation properties in accordance with embodiments herein.

FIG. 6 illustrates a block diagram illustrating an example ultrasound system that supports variable speed of sound beamforming based on automatic detection of tissue type and density characteristics in accordance with embodiments herein.

### DETAILED DESCRIPTION

Various implementations in accordance with the present disclosure may be directed to variable speed of sound beamforming based on automatic detection of tissue type in ultrasound imaging.

The foregoing summary, as well as the following detailed description of certain embodiments will be better understood when read in conjunction with the appended drawings. To the extent that the Figures illustrate diagrams of the functional blocks of various embodiments, the functional blocks are not necessarily indicative of the division between hardware circuitry. Thus, for example, one or more of the functional blocks (e.g., processors or memories) may be implemented in a single piece of hardware (e.g., a general purpose signal processor or a block of random access memory, hard disk, or the like) or multiple pieces of hardware. Similarly, the programs may be stand-alone programs, may be incorporated as subroutines in an operating system, may be functions in an installed software package, and the like. It should be understood that the various embodiments are not limited to the arrangements and instrumentality shown in the drawings. It should also be understood that the embodiments may be combined, or that other embodiments may be utilized and that structural, logical and electrical changes may be made without departing from the scope of the various embodiments. The following detailed description is, therefore, not to be taken in a limiting sense, and the scope of the present invention is defined by the appended claims and their equivalents.

As used herein, an element or step recited in the singular and proceeded with the word “a” or “an” should be understood as not excluding plural of said elements or steps, unless such exclusion is explicitly stated. Furthermore, references to “an embodiment,” “one embodiment,” “a representative embodiment,” “an example embodiment,” “various embodiments,” “certain embodiments,” and the like are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional elements not having that property.

In various implementations in accordance with the present disclosure, ultrasound imaging systems (such as, e.g., the medical imaging system, when implemented as ultrasound imaging system) may be configured to support and/or utilized variable speed of sound beamforming based on automatic detection of tissue type and/or density. In this regard, existing ultrasound systems typically utilize, and are configured to operate based on single and universal audio speed (e.g., 1540 m/s), irrespective of actual tissue densities in local regions of an ultrasound image. However, sound may have different speeds in different tissue types (e.g., muscle, fat, skin, connective tissue, etc.) and/or densities, and ultrasound imaging may be improved and optimized by using and/or accounting for such different sound speeds (that is, the actual local speed corresponding to each particular type of tissue). Accordingly, in various example implementations, local speeds of sound may be determined or estimated, and then utilized to adjust to beamforming parameters utilized during beamforming in connection with producing ultrasound imaging.

The term “class” refers to the classification of the tissue type and density characteristic, were each class is uniquely associated with a tissue type and/or density characteristic. For example, separate classes may be provided for hard fat region, normal fat region, soft fat region, hard liver region, normal liver region, soft liver region, hard kidney region, normal kidney region and soft kidney region. It is recognized that numerous other classes may be utilized for different tissue types. It is also recognized that the density characteristics may be divided into different, more or fewer classes than hard, normal and soft.

Embodiments herein may be implemented in connection with the structure and functions described in one or more of the following published patent applications:

U.S. application Ser. No. 14/088,068, titled “METHOD AND SYSTEM FOR LESION DETECTION IN ULTRASOUND IMAGES”, filed on Nov. 22, 2013;

U.S. application Ser. No. 15/367,275, titled “AUTOMATED SEGMENTATION USING DEEP LEARNED PRIORS”, filed on Dec. 2, 2016;

U.S. application Ser. No. 15/374,420, titled “VARIABLE SPEED OF SOUND BEAMFORMING BASED ON AUTOMATIC DETECTION OF TISSUE TYPE IN ULTRASOUND IMAGING”, filed on Dec. 9, 2016;

U.S. application Ser. No. 15/471,515, titled “METHOD AND SYSTEM FOR ADJUSTING AN ACQUISITION FRAME RATE FOR MOBILE MEDICAL IMAGING”, filed on Mar. 28, 2017;

U.S. application Ser. No. 15/587,568, titled “METHODS AND SYSTEMS FOR ACQUISITION OF MEDICAL IMAGES FOR AN ULTRASOUND EXAM”, filed on May 5, 2017;

U.S. application Ser. No. 15/900,386, titled “METHODS AND SYSTEMS FOR HIERARCHICAL MACHINE LEARNING MODELS FOR MEDICAL IMAGING”, filed on Feb. 20, 2018;

All of the published patents, patent applications and other publications referenced above, and hereafter, are expressly incorporated herein by reference in their entirety. The ultrasound data sets obtained in connection with embodiments herein may correspond to various types of ultrasound information (e.g., B mode, power Doppler, Doppler, strain, two-dimensional, three-dimensional, four dimensional or otherwise), as described herein and as described in the patents, patent applications and other publications referenced and incorporated herein.

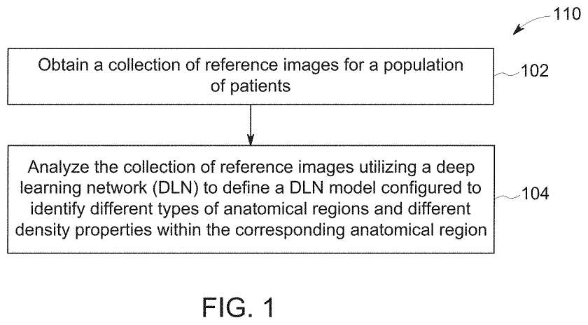

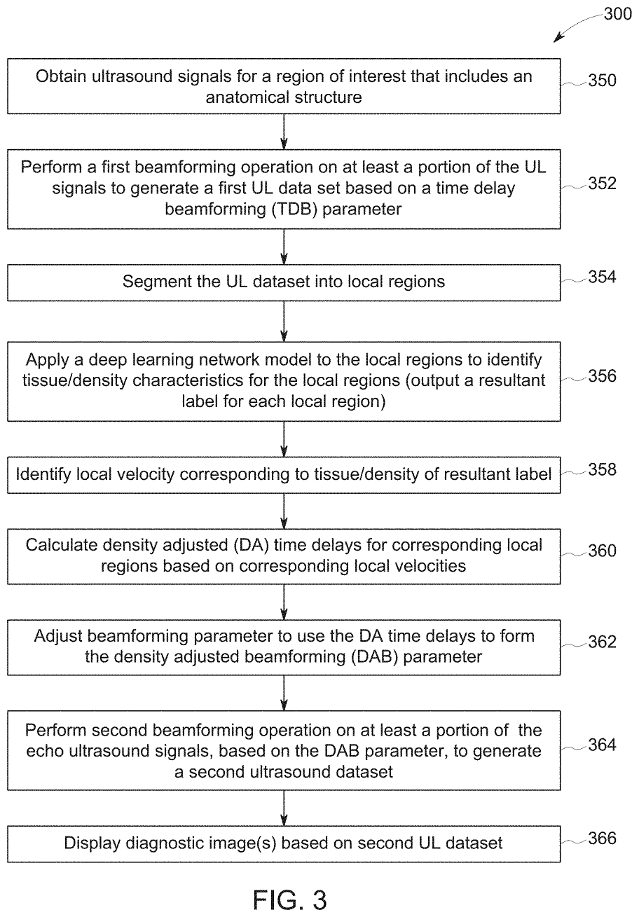

FIGS. 1 and 3 illustrate a process for managing beamforming parameters based on tissue characteristics in accordance with embodiments herein. The process of FIG. 1 corresponds to a learning segment 110, that may be performed separate in time from an implementation segment 300 (FIG. 3). The learning and implementation segments 110, 300 may be implemented on a single system and/or distributed between multiple systems. For example, the learning segment 110 may be implemented on a server and/or other network based system, while the implementation segment 300 may be implemented at individual ultrasound systems. The learning and implementation segments 110, 300 may be implemented generally contemporaneous with one another and/or at diverse points in time. Additionally or alternatively, the learning segment 110 may be iteratively updated over time before, during and/or after implementation segments 300 are implemented at a common or separate ultrasound system.

Beginning with the learning segment 110 of FIG. 1, at 102, one or more processors obtain a collection of reference images for a population of patients. The collection of reference images may be iteratively updated over time. The reference images represent ultrasound images that are obtained from a patient population of different types of tissue for one or more anatomical regions. In connection with each anatomical region, different reference images are collected for patients exhibiting different tissue characteristics within the common and/or around the corresponding anatomical region. For example, a first subset of the collection may correspond to ultrasound images of livers from multiple patients who exhibit different density properties within or around the liver. For example, the density properties within or around the liver may be classified into hard, normal and soft liver regions. A portion of the patients may have a fatty liver, while others have a normal liver, and yet others have hardening of the liver. The classification may be based solely on an interior composition of the liver, based on a combination of a composition of the liver and a surrounding region, and/or based solely on an exterior composition of the region surrounding the liver. As a further example, a second subset of the collection may correspond to ultrasound images of kidneys from multiple patients who exhibit different density properties within or around the kidney. For example, the density properties within or around the kidney may be classified into hard, normal and soft liver regions. As further examples, another subset of the reference images may correspond to fat regions that exhibit different density properties (e.g., a hard fat region, a normal fat region, a soft fat region). Additionally or alternatively, other subsets of the reference images correspond to other anatomical regions, such as myocardium tissue, air, lungs, water, the brain, skull bone, interior bones and the like, where ultrasound images are captured from patients exhibiting different density properties in connection with a corresponding anatomical region.

At 104, the one or more processors analyze the collection of reference images utilizing a deep learning network (DLN) to define a DLN model configured to identify different types of anatomical regions and different density properties within the corresponding anatomical region. Once trained, the DLN model distinguishes between liver, kidneys, myocardium tissue, air, lungs, water, the brain, skull bone, interior bones and the like. Additionally, the DLN model distinguishes between different density properties within a single type of anatomical structure, for example to distinguish between hard, normal or soft liver regions; hard, normal or soft fat regions; hard, normal or soft kidney regions and the like. The DLN model is saved, such as on a local ultrasound system, server or other network. The DLN model may then be distributed to multiple ultrasound systems for subsequent real-time use during an examination. Additionally or alternatively, the DLN model may be periodically updated based on new reference images.

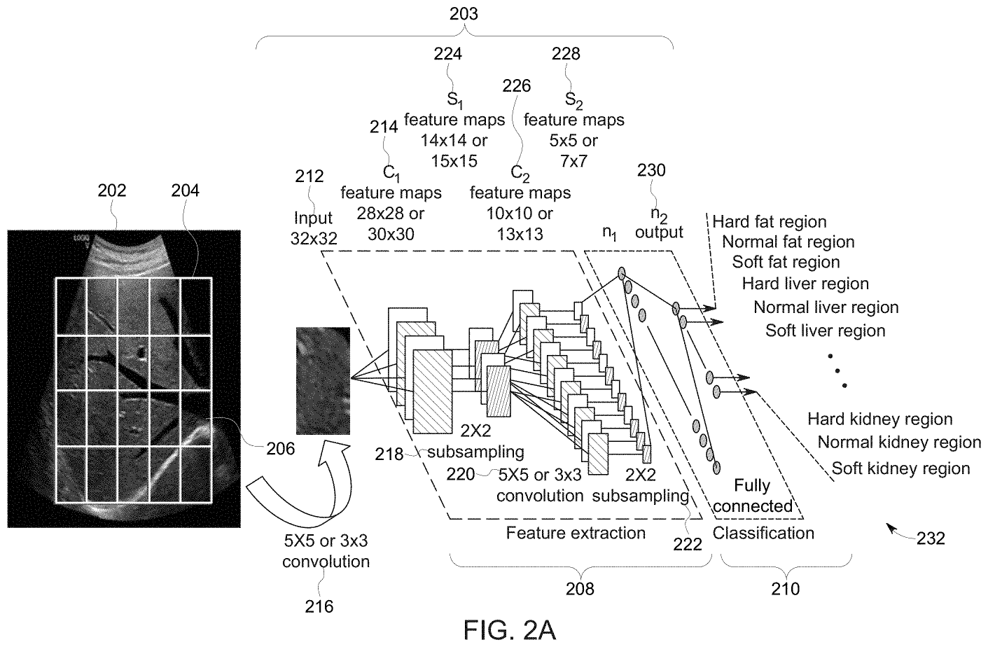

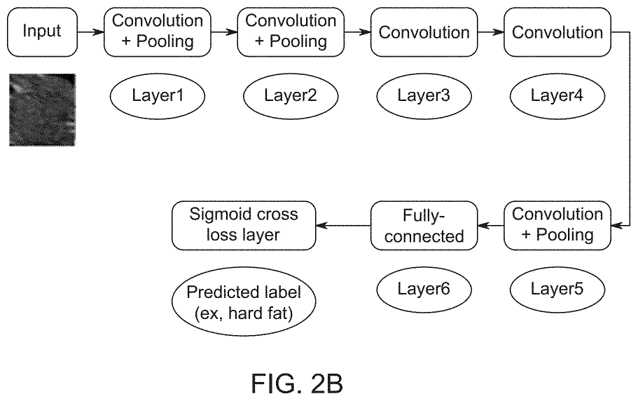

FIG. 2A illustrates a graphical representation of a process in which the DLN model is built. The one or more processors obtain a collection of reference images for a population of patients. In FIG. 2A, a reference image 202 is illustrated as a B-mode image of a region of interest that includes one or more anatomical structures. The one or more processors analyze the reference image 202 utilizing a deep learning network (DLN) 203 to define a DLN model configured to identify different types of anatomical regions and different density properties within the corresponding anatomical region. For example, the deep learning network 203 builds a connection layer linking different features to probabilities that an input local region is liver, kidneys, myocardium tissue, air, lungs, water, the brain, skull bone, interior bones and the like. Additionally, the deep learning network 203 distinguishes between different density properties within a single type of anatomical structure, for example to distinguish between hard, normal or soft liver regions; hard, normal or soft fat regions; hard, normal or soft kidney regions and the like.

The reference image 202 is segmented, such as utilizing a matrix 204, into local regions 206. The shapes of the local regions 206 may vary and may differ in size from one another. Each or a select portion of the local regions 206 are applied separately as individual inputs 212 to the neural network 203. The local region 206 includes an array of pixels (e.g., a 32×32 array).

Each of the local regions 206 is individually processed by the deep learning neural network 203 to build feature maps and links between the feature maps and tissue types and/or density characteristics associated with the local region 206. For example, the neural network may represent a convolutional neural network or other type of neural network that is useful in image recognition and classification. The convolutional neural network 203 is built through four primary operations, namely one or more convolution, nonlinearity, pooling or sub-sampling and classification operations.

A convolution function 216, such as a 5×5 matrix or kernel, is applied to the pixel array within the local region 206. Optionally, the convolution function 216 may be formed from a matrix or kernel of different dimension, including but not limited to a 3×3 matrix. The output of the convolution is pooled (e.g., sub-sampled) at 218 to form a first feature map 214 (also referred to as a convolved feature map). By way of example, the first feature map 214 may represent a 28×28 array of features corresponding to the convolved sub-sampled output of the original pixel array. Optionally, the feature map 214 may vary in size, including but not limited to a 30×30 feature map, 15×15 feature map, 14×14 feature map, 7×7 feature map, 5×5 feature map and the like. The 5×5 kernel is slid over the pixel array of the local region 206 and a dot product is computed at each position to form the feature map 214. The convolution function 216 preserves a spatial relation between the pixels of the local region 206 while learning image features for small areas/squares of input data/pixels. The first feature map 214 is then processed by a second convolution function 220 to form a second feature map 224. By way of example, the second convolution function 220 may utilize a 5×5 convolution kernel, and the set of second feature maps 224, each may include a 14×14 array of features. Next, the set of second feature maps 224 is sub-sampled at 222 to form a set of third feature maps 226. By way of example, the sub-sampling at 222 may form a set of 10×10 features map 228.

The sub-sampling operations reduce the dimensionality of each feature map, while retaining information of interest. Sub-sampling may be performed in different manners, such as by identifying maximums, averages, sums and the like. For example, in a maximum sub-sampling operation, a spatial neighborhood may be defined, with the largest element from the neighborhood forming the single output (e.g., converting a 2×2 matrix of pixels to a single pixel having the maximum value from the 2×2 matrix).

Optionally, an additional operation, namely a non-linearity activation function, may be applied after one or more convolution operation and before the corresponding sub-sampling operation. For example, the nonlinearity activation function may be defined by a rectified linear unit that is applied as an element wise operations (e.g., per pixel). As one example, the nonlinearity activation function may replace negative pixel values in the corresponding feature map with zeros or another non-negative value. Optionally, the nonlinearity activation function may be omitted.

Once one or more sets of feature maps of desired dimension are generated, the feature maps are output at 230 from the feature extraction section 208 and are passed to a classification section 210. The output of the feature extraction section 208 represents high-level features of the ultrasound data in the original local region 206. The classification section 210 then builds the DLN model. The DLN model includes a connected layer that uses the high level features from the feature extraction section 208 for classifying the input image local region into various classes.

The connected layer performs an operation in which the input (e.g., the feature map at 230) is “flattened” into a feature vector. The feature vector is passed through a network of “neurons” to predict the output probability. The feature vector is then passed through multiple dense layers, at each of which the feature vector is multiplied by the layer weight, summed with a corresponding bias and passed through a nonlinearity function. An output layer generates a probability for each class that is potentially in the input local region.

It is understood that the examples for convolution functions, feature maps, pooling functions and the like, provide nonlimiting examples of the sizes of the corresponding functions and maps through all layer of DLN model. The functions and maps may vary widely in size, including but not limited to 28×28, 30×30, 15×15, 14×14, 13×13, 10×10, 7×7, 5×5, 3×3 and the like.

FIG. 2B illustrates an alternative graphical representation of a process in which the DLN model is built in accordance with an embodiment herein. In FIG. 2B, a local region is provided as an input which is passed through multiple layers. Layers 1 and 2 apply convolution and pooling, while layers 3 and 4 only apply convolution. Layer 5 applies convolution and pooling, while layer 6 defines a fully connected relation.

FIG. 2B also illustrates a layer 7 that is utilized to measure an accuracy of a network in predicting a particular class for a input (e.g., local region). The layer 7 may utilize a sigmoid cross loss function to predict output classes. In the present example, the sigmoid cross entropy loss is applied, each local region (image patch) is provided as an input. The local regions are annotated with a vector of ground-truth label probabilities pᵢ, where the vector has a length C corresponding to the number of class available (e.g., the number of potential combinations of tissue type and density characteristic). The neural network model 203 is trained by minimizing the following loss objective equation:

$${E}_{e} = {\sum }_{i = 1}^{N} {\sum }_{c = 1}^{C} [ {p}_{i} log ( \hat{{p}_{i}} ) + ( 1 - {p}_{i} ) log ( 1 - \hat{{p}_{i}} ) ] + γ {‖ W ‖}_{2}$$

$${w}_{i} ← {w}_{i} + Δ {w}_{i} , \\ Δ {w}_{i} = - η \frac{\partial E}{\partial {w}_{i}}$$

$$Updating on weight parameters$$

In the foregoing loss objective equation, γ∥W∥₂ is the L2 regularization on weight W of the DLN model 203, while γ is a regularization parameter. The probability vector \hat{p}_{ι} is obtained by applying the sigmoid function to each of the C class outputs of the DLN model in FIG. 2B.

Once the neural network model 203 is trained, the neural network model 203 is stored for later use during implementation in connection with individual patient ultrasound scans.

FIG. 3 illustrates the implementation segment 300 of a process for managing a beamforming operation based on tissue/density characteristics of local regions in a region of interest in accordance with embodiments herein. The operations of FIG. 3 may be performed in real-time during an ultrasound examination while a patient is present and being actively scanned. Additionally or alternatively, the operations may be performed at different points in time, such as after the raw ultrasound signals are acquired. As another example, the operations of FIG. 3 may be performed on “historic” non-beamformed ultrasound signals that were collected in the past.

At 350, a probe is operable to transmit ultrasound signals and receive echo ultrasound signals from a region of interest (ROI) during a first scan of the ROI. The first scan may cover a single slice, a volume, or portions thereof. For example, the first scan may represent a scout or calibration scan that is performed at a common resolution or lower resolution than utilized during a diagnostic scan (e.g., at 364). A scout scan may collect ultrasound signals along scan lines that are spaced apart or separated from one another more than in a diagnostic imaging scan. Additionally or alternatively, when scanning a volume, the scout or calibration scan may scan slices of the volume spaced apart from adjacent scan slices by a distance (slice-to-slice) greater than a slice-to-slice distance in a diagnostic imaging scan.

At 352, processing circuitry is operable to perform a first beamforming operation on at least a portion of the echo ultrasound signals to generate a first ultrasound dataset, corresponding to a first ultrasound image, based on a time delay beamforming (TDB) parameter. The TDB parameter includes a first set of initial time delay values and weights. Optionally, the processing circuitry may display the first ultrasound image on the display of an ultrasound system, workstation, laptop computer, portable device (e.g., smart phone, tablet device, etc.) and the like. The first ultrasound image may correspond to a medical diagnostic image of the region of interest, a medical diagnostic image of a portion of the region of interest, a scout or calibration scan of the region of interest or a portion of the region of interest, and the like. The first ultrasound image may correspond to a single slice through a volumetric region of interest and/or a three-dimensional volumetric image of a volumetric region of interest. The first ultrasound image may be presented in any known ultrasound format, such as B mode, color Doppler, 3-D imaging, 4D imaging and the like.

At 354, the processing circuitry is operable to segment the first ultrasound dataset into local regions. Each local region is a local image patch. The segmentation process may be performed entirely automatically based on various segmentation algorithms. By way of example, the segmentation may be based upon identification of anatomic features or characteristics within the first ultrasound image. Additionally or alternatively, the segmentation may be based on other segmentation techniques, such as seed-based, border discrimination and the like.

At 356, the processing circuitry is operable to apply the deep learning network (DLN) model, determined through the process of FIGS. 1, 2A and 2B to the local regions to identify tissue type and/or density characteristics associated with corresponding portions of the ROI in the associated local regions. The DLN model classifies the local regions to correspond to one of at least two different types of tissue. By way of example, the types of tissue include at least two of air, lung, fat, water, brain, kidney, liver, myocardium, or bone. The DLN model identifies the tissue type and/or density characteristic and outputs at least one resultant label for each associated local region. For example, the resultant label may name hard fat as the tissue type and density, along with a probability that the resultant label is correct. The DLN model outputs a resultant label for each local region that is input. As a nonlimiting example, when an ultrasound data set is divided into a 32×32 matrix of image patches, each of which corresponds to a local region, the DLN model will output resultant labels, each of which corresponds to an individual image patch.

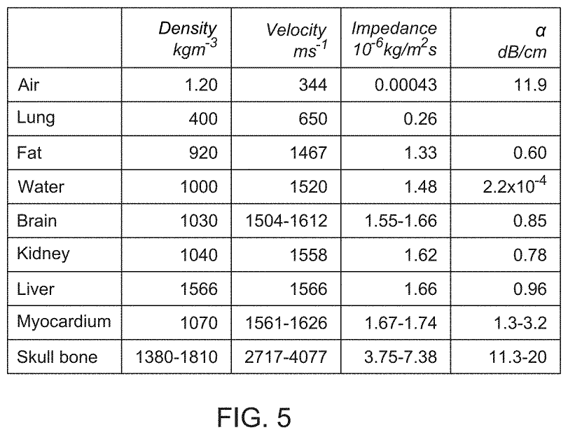

At 358, the processing circuitry is operable to identify one or more local velocities corresponding to the local regions (image patches) based on the resultant labels and a density table. FIG. 5 illustrates a density table designating different tissue types, along with corresponding densities, velocities, impedances and attenuation properties. By way of example, when a resultant label designates a local region to represent kidney, the local velocity would be determined to equal 1558 m/s. The table in FIG. 5 illustrates a single density associated with each type of tissue. Optionally, a single type of tissue may be further divided into a group of densities, each of which has a separate corresponding velocity. For example, separate velocities may be assigned for hard, normal and soft kidneys, while separate velocities are signed for hard, normal and soft livers, and the like.

In the present example, one resultant label and one local velocity is assigned to each local region (image patch). Additionally or alternatively, multiple resultant labels and multiple local velocities may be assigned to a single local region. For example, probabilities of two or more resultant labels may be within a range of one another (e.g., within 20% of one another). In the event that multiple resultant labels have similar probabilities, the operation at 358 may determine corresponding local velocities and form a mathematical combination thereof, such as an average, mean and the like. Alternatively, when multiple resultant labels are identified for a single local region, the operation at 358 may select one of the corresponding local velocities, such as the highest, lowest or median local velocity for a group of resultant labels.

At 360, the processing circuitry is operable to calculate density adjusted (DA) time delays for the corresponding local regions based on the corresponding local velocities. For example, the DA time delays may be calculated based on the following equation. The basic concept to use initial time-delay values for beamforming is as follows:

h(t)=Σₘ₌₀^{M-1}wₘxₘ(t−τₘ)
xₘ: output signal of each array element,
   wₘ: dynamically updated weighting,
τₘ: desired dynamically updated delay.

$${τ}_{m} = \frac{{l}_{m}}{c}$$

delay comes from the propagation time for each array element to focal point with pre-determined sound speed.

$${τ}_{m}^{′} = \frac{{l}_{m}}{{c}^{′}} ,$$

new delay comes from the propagation time for each array element to focal point with the right sound speed resulting from the attenuation of tissue composition. At 360, the new DA time delay values are utilized for DA beamforming as follows:

h′(t)=τₘ₌₀^{M-1}wₘxₘ(t−τ′ₘ)

xₘ: output signal of each array element, wₘ: dynamically updated weighting,
τ′ₘ: desired dynamically updated new delay.
   Once the DA time delays are calculated, flow advances to 362.

At 362, the processing circuitry is operable to adjust the beamforming parameters, based on the set of DA time delays for the corresponding local regions, to form a density adjusted beamforming (DAB) parameter. The TDB parameter includes set of first or initial time delay values associated with a common reference density and are used at 352. The processing circuitry is operable to adjust the TDB parameter to form the DAB parameter by changing the set of the first time delay values to a set of second time delay values associated with a predicted or actual densities corresponding to the tissue/density characteristics identified by the DLN model. A second time delay value is determined based on a propagation time from an array element of the probe to a focal point in the ROI utilizing a predicted speed of sound that is determined based on the density characteristics identified by the DLN model.

At 364, the processing circuitry is operable to perform a second beamforming operation on at least a portion of the echo ultrasound signals, based on the DAB parameter, to generate a second ultrasound dataset. The TDB parameter and DAB parameter include different first and second delays that are utilized during the first and second beamforming, respectively, in connection with a common segment of the ROI. Optionally, the first and second beamforming are performed on a common portion of the echo ultrasound signals. Optionally, the first and second beamforming operations may be performed on different echo ultrasound signals. For example, the first beamforming operation (at 352) may be performed during a first scan such as during a calibration scan. Thereafter, once the DAB parameter is corrected based on a particular patient's tissue type(s) within the ROI, a second diagnostic imaging scan is performed during a full patient examination. Optionally, the first scan may be saved from one patient visit to a physician and the DAB parameter may be used during later patient visits.

Optionally, the probe may perform first and second scans of the ROI (at 352 and 364), during which first and second sets of the echo ultrasound signals are received. Optionally, the first scan is performed before the first beamforming operation, while the second scan is performed after the first beamforming operation and before the second beamforming operation.

At 366, the processing circuitry is operable to display one or more diagnostic images based on the second ultrasound dataset. Optionally, the processing circuitry may display the second ultrasound image on the display of an ultrasound system, workstation, laptop computer, portable device (e.g., smart phone, tablet device, etc.) and the like. The second ultrasound image may correspond to a medical diagnostic image of the region of interest, a medical diagnostic image of a portion of the region of interest, a scout or calibration scan of the region of interest or a portion of the region of interest, and the like. The second ultrasound image may correspond to a single slice through a volumetric region of interest and/or a three-dimensional volumetric image of a volumetric region of interest. The second ultrasound image may be presented in any known ultrasound format, such as B mode, color Doppler, 3-D imaging, 4D imaging and the like.

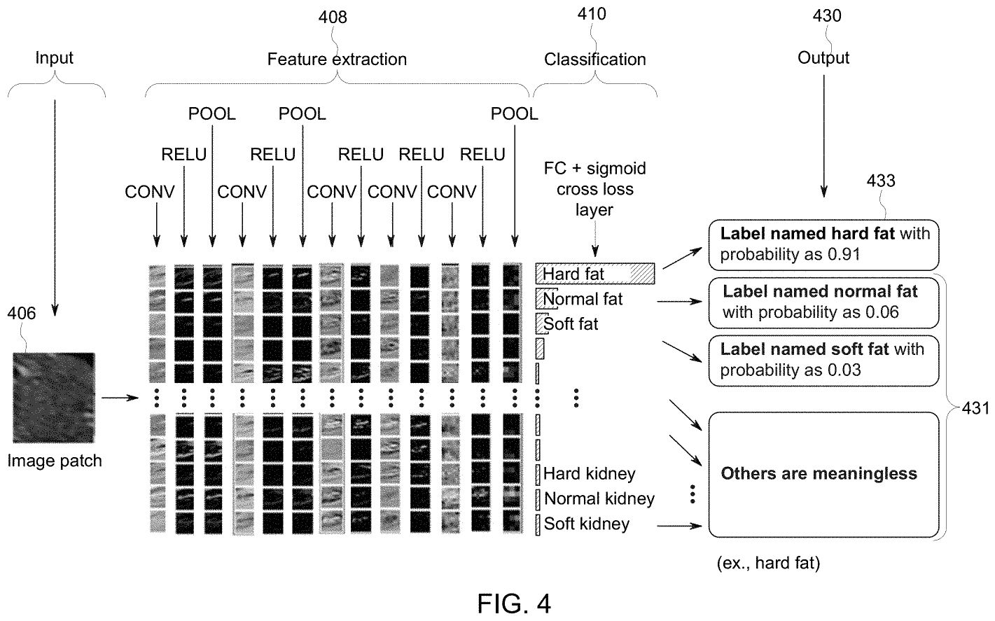

FIG. 4 illustrates a block diagram of an implementation that applies a DLN model in accordance with an embodiment herein. The DLN model was trained as explained above to generate candidate labels associated with some or all available classes, were each candidate label is assigned a probability that the corresponding candidate label in fact corresponds to the input. The DLN model assigns one or a small subset of the candidate labels to have a high probability, where the candidate label(s) with the highest probability is then designated as the resultant label(s).

As noted herein, the ultrasound data set for a current scan is segmented into local regions, such as local region 406. The local region 406 is passed to the feature extraction section 408, in which the various operations are performed as described above, including convolutions, pooling and nonlinearity functions. The feature extraction section 408 generates one or more feature maps that are passed to the classification section 410 which performs feature classification in connection with identifying a class corresponding to tissue type and/or a density characteristic. The classification section 410 provides an output 430. The output 430 may include one or more labels that designate one or more classes, along with a corresponding probability that the class designation is correct. The output 430 may not include every potential class of tissue type and/or density characteristic, but instead merely include the subset of classes that may potentially correspond to the input local region 406. In the present example, the classification section 410 provides an output 430 that includes a resultant label 433 indicating that a probability of 0.91 exist that the input image patch 406 represents “hard fat”. Optionally, the output 430 may also include a group of candidate labels 431 that include probabilities associated with some or all of the other potential classes. For example, the candidate labels 431 may include candidate labels indicating that probabilities of 0.06 and 0.03 exist that the input image patch 406 represents normal fat and soft fat, respectively. The remaining candidate labels are assigned even smaller probabilities and thus are considered meaningless. Accordingly, the output 430 designates that the expected label/class for the input image patch 406 is hard fat.

### Ultrasound System

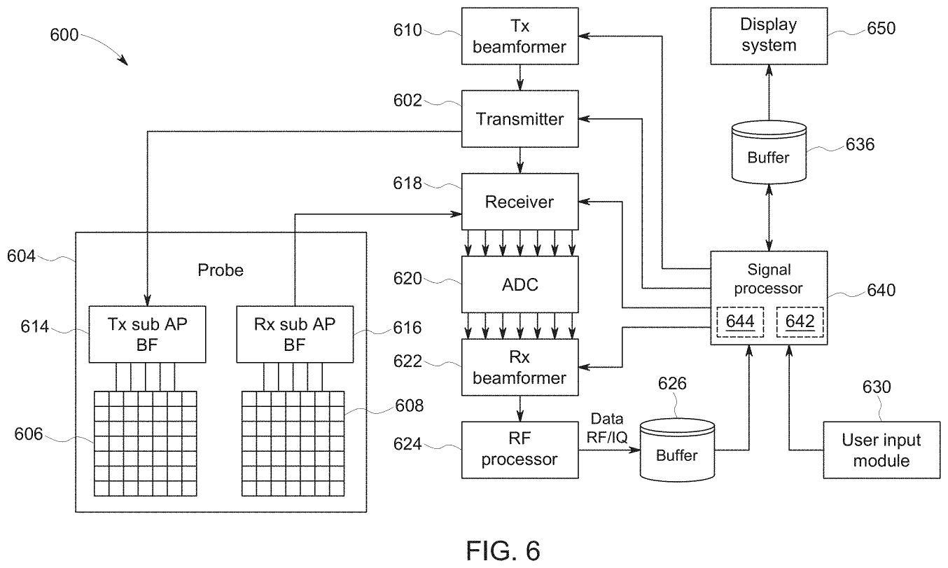

FIG. 6 is a block diagram illustrating an example ultrasound system that supports variable speed of sound beamforming based on automatic detection of tissue type and density characteristics in accordance with embodiments herein. The ultrasound system 600 may comprise suitable components (physical devices, circuitry, etc.) for providing ultrasound imaging. The ultrasound system 600 comprises, for example, a transmitter 602, an ultrasound probe 604, a transmit beamformer 610, a receiver 618, a receive beamformer 622, a RF processor 624, a RF/IQ buffer 626, a user input module 630, a signal processor 640, an image buffer 636, and a display system 650.

The transmitter 602 may comprise suitable circuitry that may be operable to drive the ultrasound probe 604. The transmitter 602 and the ultrasound probe 604 may be implemented and/or configured for one-dimensional (1D), two-dimensional (2D), three-dimensional (3D), and/or four-dimensional (4D) ultrasound scanning. The ultrasound probe 604 may comprise a one-dimensional (1D, 5.25D, 5.5D or 5.75D) array or a two-dimensional (2D) array of piezoelectric elements. For example, as shown in FIG. 6, the ultrasound probe 604 may comprise a group of transmit transducer elements 606 and a group of receive transducer elements 608, that normally constitute the same elements. The transmitter 602 may be driven by the transmit beamformer 610.

The transmit beamformer 610 may comprise suitable circuitry that may be operable to control the transmitter 602 which, through a transmit sub-aperture beamformer 614, drives the group of transmit transducer elements 606 to emit ultrasonic transmit signals into a region of interest (e.g., human, animal, underground cavity, physical structure and the like). In this regard, the group of transmit transducer elements 606 can be activated to transmit ultrasonic signals. The ultrasonic signals may comprise, for example, pulse sequences that are fired repeatedly at a pulse repetition frequency (PRF), which may typically be in the kilohertz range. The pulse sequences may be focused at the same transmit focal position with the same transmit characteristics. A series of transmit firings focused at the same transmit focal position may be referred to as a “packet.”

The transmitted ultrasonic signals may be back-scattered from structures in the object of interest, like tissue, to produce echoes. The echoes are received by the receive transducer elements 608. The group of receive transducer elements 608 in the ultrasound probe 604 may be operable to convert the received echoes into analog signals, undergo sub-aperture beamforming by a receive sub-aperture beamformer 616 and are then communicated to the receiver 618.

The receiver 618 may comprise suitable circuitry that may be operable to receive and demodulate the signals from the probe transducer elements or receive sub-aperture beamformer 616. The demodulated analog signals may be communicated to one or more of the plurality of A/D converters (ADCs) 620.

Each plurality of A/D converters 620 may comprise suitable circuitry that may be operable to convert analog signals to corresponding digital signals. In this regard, the plurality of A/D converters 620 may be configured to convert demodulated analog signals from the receiver 618 to corresponding digital signals. The plurality of A/D converters 620 are disposed between the receiver 618 and the receive beamformer 622. Notwithstanding, the disclosure is not limited in this regard. Accordingly, in some embodiments, the plurality of A/D converters 620 may be integrated within the receiver 618.

The receive beamformer 622 may comprise suitable circuitry that may be operable to perform digital beamforming processing to, for example, sum the delayed channel signals received from the plurality of A/D converters 620 and output a beam summed signal. The resulting processed information may be converted back to corresponding RF signals. The corresponding output RF signals that are output from the receive beamformer 622 may be communicated to the RF processor 624. In accordance with some embodiments, the receiver 618, the plurality of A/D converters 620, and the beamformer 622 may be integrated into a single beamformer, which may be digital.

The RF processor 624 may comprise suitable circuitry that may be operable to demodulate the RF signals. In some instances, the RF processor 624 may comprise a complex demodulator (not shown) that is operable to demodulate the RF signals to form In-phase and quadrature (IQ) data pairs (e.g., B-mode data pairs) which may be representative of the corresponding echo signals. The RF (or IQ) signal data may then be communicated to an RF/IQ buffer 626.

The RF/IQ buffer 626 may comprise suitable circuitry that may be operable to provide temporary storage of output of the RF processor 624 (e.g., the RF (or IQ) signal data, which is generated by the RF processor 624).

The user input module 630 may comprise suitable circuitry that may be operable to enable obtaining or providing input to the ultrasound system 600, for use in operations thereof. For example, the user input module 630 may be used to input patient data, surgical instrument data, scan parameters, settings, configuration parameters, change scan mode, and the like. In an example embodiment, the user input module 630 may be operable to configure, manage and/or control operation of one or more components and/or modules in the ultrasound system 600. In this regard, the user input module 630 may be operable to configure, manage and/or control operation of transmitter 602, the ultrasound probe 604, the transmit beamformer 610, the receiver 618, the receive beamformer 622, the RF processor 624, the RF/IQ buffer 626, the user input module 630, the signal processor 640, the image buffer 636, and/or the display system 650.

The signal processor 640 may comprise suitable circuitry that may be operable to process the ultrasound scan data (e.g., the RF and/or IQ signal data) and/or to generate corresponding ultrasound images, such as for presentation on the display system 650. The signal processor 640 is operable to perform one or more processing operations according to a plurality of selectable ultrasound modalities on the acquired ultrasound scan data. In some instances, the signal processor 640 may be operable to perform compounding, motion tracking, and/or speckle tracking. Acquired ultrasound scan data may be processed in real-time (e.g., during a B-mode scanning session), as the B-mode echo signals are received. Additionally or alternatively, the ultrasound scan data may be stored temporarily in the RF/IQ buffer 626 during a scanning session and processed in less than real-time in a live or off-line operation.

In operation, the ultrasound system 600 may be used in generating ultrasonic images, including two-dimensional (2D), three-dimensional (3D), and/or four-dimensional (4D) images. In this regard, the ultrasound system 600 may be operable to continuously acquire ultrasound scan data at a particular frame rate, which may be suitable for the imaging situation in question. For example, frame rates may range from 50-70 but may be lower or higher. The acquired ultrasound scan data may be displayed on the display system 650 at a display-rate that can be the same as the frame rate, or slower or faster. An image buffer 636 is included for storing processed frames of acquired ultrasound scan data that are not scheduled to be displayed immediately. Preferably, the image buffer 636 is of sufficient capacity to store at least several seconds' worth of frames of ultrasound scan data. The frames of ultrasound scan data are stored in a manner to facilitate retrieval thereof according to its order or time of acquisition. The image buffer 636 may be embodied as any known data storage medium.

In some instances, the ultrasound system 600 may be configured to support grayscale and color based operations. For example, the signal processor 640 may be operable to perform grayscale B-mode processing and/or color processing. The grayscale B-mode processing may comprise processing B-mode RF signal data or IQ data pairs. For example, the grayscale B-mode processing may enable forming an envelope of the beam-summed receive signal by computing the quantity (I²+Q²)^{1/2}. The envelope can undergo additional B-mode processing, such as logarithmic compression to form the display data. The display data may be converted to X-Y format for video display. The scan-converted frames can be mapped to grayscale for display. The B-mode frames that are provided to the image buffer 636 and/or the display system 650. The color processing may comprise processing color based RF signal data or IQ data pairs to form frames to overlay on B-mode frames that are provided to the image buffer 636 and/or the display system 650. The grayscale and/or color processing may be adaptively adjusted based on user input (e.g., a selection from the user input module 630), for example, for enhance of grayscale and/or color of particular area.

In some instances, ultrasound imaging may include generation and/or display of volumetric ultrasound images (that is where objects (e.g., organs, tissues, etc.) are displayed three-dimensional 3D). In this regard, with 3D (and similarly 4D) imaging, volumetric ultrasound datasets may be acquired, comprising voxels that correspond to the imaged objects. This may be done, e.g., by transmitting the sound waves at different angles rather than simply transmitting them in one direction (e.g., straight down), and then capture their reflections back. The returning echoes (of transmissions at different angles) are then captured, and processed (e.g., via the signal processor 640) to generate the corresponding volumetric datasets, which may in turn be used (e.g., via a 3D rendering module 642 in the signal processor 640) in creating and/or displaying volume (e.g., 3D) images, such as via the display system 650. This may entail use of particular handling techniques to provide the desired 3D perception.

For example, volume rendering techniques may be used in displaying projections (e.g., 6D projections) of the volumetric (e.g., 3D) datasets. In this regard, rendering a 5D projection of a 3D dataset may comprise setting or defining a perception angle in space relative to the object being displayed, and then defining or computing necessary information (e.g., opacity and color) for every voxel in the dataset. This may be done, for example, using suitable transfer functions for defining RGBA (red, green, blue, and alpha) value for every voxel.

In various implementations in accordance with the present disclosure, the ultrasound system 600 may be configured to support variable speed of sound beamforming based on automatic detection of tissue type in ultrasound imaging. In particular, the ultrasound system 600 may be configured to assess the area being imaged to identify different types of tissue in it, and then perform ultrasound imaging based on actual local speeds of sound corresponding to each of the recognized types of tissue. In this regard, as noted above, sound may have different speed in different tissue types (e.g., muscle, fat, skin, connective tissue, etc.). Thus, quality of ultrasound images may be enhanced by using and/or accounting for the actual local speed corresponding to each particular type of tissue. In this regard, in ultrasound imaging, the image quality, in particular lateral resolution and contrast, is dependent on, at least in part, the transmit and receive beamforming process and data obtained based thereon.

Improving particular lateral resolution and contrast, and thus overall image quality, may be achieved based on knowledge (and use) of local sound speed in the imaged area. Existing systems and/or methods may be implemented in accordance with the incorrect assumption of a universal speed of sound in the human body, resulting in inferior image quality. In this regard, ultrasound beamforming processes in existing systems and methods are configured (e.g., use time delays adjusted based on) a single constant speed of sound, typically the universal sound speed of 1540 m/s. However, different tissues have varying speeds of sound due to their varying mechanical properties (e.g., 1450 m/s in fat, 1613 m/s in skin and connective tissue, etc.). The variations in speed of sound between the presumed universal sound speed and the actual local sound speed(s) may lead to incorrect focusing and/or increased clutter in generated images.

Thus, by knowing and using speed of sound accurately and locally in ultrasound imaging (e.g., the beamforming process) based on the actual local sound speeds for the tissue types in the imaged area, ultrasound image quality can be improved. For example, the transmit and receive beamforming process in the ultrasound system 600 may be configured to accommodate local variations in sound speed. Configuring ultrasound imaging (particularly, e.g., beamforming process used during such ultrasound imaging) in this manner would produce a perfectly focused image with higher contrast and resolution. Further, the geometry of the image may be rectified. This allows for more precise measurements. This may be particularly pertinent with particular types of patients (e.g., obese patients) and/or in exams of particular areas (e.g., breast imaging).

In an example implementation, an ultrasound system (e.g., the ultrasound system 600) may be configured to determine or estimate local speed of sound (e.g., via a sound speed control module 644 in the signal processor 640). These local speeds of sound may then be used in optimize the ultrasound imaging (e.g., in adjusting the time delay pattern in transmit and receive beamforming) that is, time delays applied to each of the received channel signals, which are summed to obtained the combined beamformed receive signal, thus improving the image quality. The sound speeds for various tissue types may be pre-stored into the system (e.g., within the signal processor 640, in a memory device (not shown), etc.), and accessed and used when needed (e.g., when corresponding types of tissues are identified during active imaging).

Detecting tissue types and/or density characteristics in this manner is advantageous because of processing speed and simplicity of implementation (requiring very minimal, if any, changes to the already utilized hardware). For example, a standard delay-and-sum beamformer can be used with this technique. By adjusting the delay times of individual channels after the image analysis has been completed, the image can be enhanced. Further, data obtained based on analysis of local features can further be used for other purposes, such as detection and segmentation of organs or pathological defects.

In an example implementation, an ultrasound system (e.g., the ultrasound system 600) may be configured to perform (e.g., via the sound speed control module 644 of the signal processor 640) analysis of local image features, to identify the tissue type and/or density characteristics in a particular part of the image, by subdividing the image into an arbitrary number of parts, which are then analyzed individually, for determining the tissue type and/or density characteristics associated with each of the parts of the image. For example, a sliding window may be used to scan different portions in the image, to identify the tissue type and/or density characteristics associated with each portion. Based on knowledge of sound speed in different tissue types, the local speed of sound can be estimated in every separate part of the image. The local features of the different tissues and/or density characteristics may be pre-programmed into the system. Alternatively, the system may be configured to determine (and store) these local features adaptively (e.g., in a separate learning process). For example, when imaging an already determined tissue type and/or density characteristics, the local features of the corresponding images may be assessed and stored for future use. The actual sound speeds associated with the different tissue types may be obtained in various ways. For example, the speed of sound for major tissue types in the human body may be well known, and as such may be pre-programmed into the systems. Further, in some instances, pre-programmed sound speeds may be tuned, such as based on actual use of the system.

In an example implementation, the adaptive adjustment of variable speed of sound beamforming based on automatic detection of tissue type and/or density characteristics may be configured as an iterative process. For example, in a first iteration, a universal speed of sound (e.g., 1540 m/s) may be used in the first iteration to construct an image using a known beamforming scheme. The local features of the beamformed image may then be analyzed, and time delays in the beamforming process may be adjusted according to the detected sound speeds. Using these adjusted time delays, an image may be obtained in a second iteration. This second image would presumably have a higher image quality. Optionally, more than two iterations can be used to further improve the image.

In an example implementation, detected local sound speeds may be used (e.g., via the signal processor 640) in segmenting images into regions with constant speed of sound. For example, by knowing the normals of region boundaries, refraction angles may be calculated. This data may then be incorporated into the beamforming process to further enhance the image.

In other example implementations, other techniques may be used for recognizing different types of tissue in areas being imaged and/or for adaptively adjusting ultrasound imaging operations to account for variation in local sound speed. For example, deterioration of image quality due to varying sound speeds in an imaged area may be addressed by omitting image analysis (e.g., including analysis of local features, as described above) and instead calculating correlation between radiofrequency (RF) signals of individual elements of the transducer. Time delays in the beamforming process may then be chosen so that these correlations are minimized. Such approach, however, requires that all element data be available to the processor. Further, this approach may require a change in the beamforming process and components used therefor. Further, a distinct feature in the image plane may be required to perform the computation, such as a point source. This may not be available in real-world imaging situations. Additionally, such approach usually assumes a single distorting layer between the tissue and the transducer (whereas with image analysis based approach, as described above, the speed of sound may be estimated in every analyzed window in the image). In another approach image analysis may be used, but with organ recognition being achieved based on machine learning techniques. In such approach knowledge about organ features (e.g., shape and texture) may be acquired, based on previously generated images, using learning algorithms, and that knowledge is then applied to new images for detection of organs (and thus type of tissue is determined from knowledge of tissue types associated with each organ). Such approach, however, requires more processing in comparison to the approach described above, which only requires analysis of local texture features and thus may be easier to implement, quicker, and less processing-intensive. In yet another approach, blind or non-blind deconvolution of an image may be used, using different kernels for different sound speeds. Such approach usually requires some way to automatically determine the image quality and to choose the best deconvolution kernel. This approach, however, may be slow and requires working globally and on the entire image.

### CLOSING STATEMENTS

It should be clearly understood that the various arrangements and processes broadly described and illustrated with respect to the Figures, and/or one or more individual components or elements of such arrangements and/or one or more process operations associated of such processes, can be employed independently from or together with one or more other components, elements and/or process operations described and illustrated herein. Accordingly, while various arrangements and processes are broadly contemplated, described and illustrated herein, it should be understood that they are provided merely in illustrative and non-restrictive fashion, and furthermore can be regarded as but mere examples of possible working environments in which one or more arrangements or processes may function or operate.

As will be appreciated by one skilled in the art, various aspects may be embodied as a system, method or computer (device) program product. Accordingly, aspects may take the form of an entirely hardware embodiment or an embodiment including hardware and software that may all generally be referred to herein as a “circuit,” “module” or “system.” Furthermore, aspects may take the form of a computer (device) program product embodied in one or more computer (device) readable storage medium(s) having computer (device) readable program code embodied thereon.

Any combination of one or more non-signal computer (device) readable medium(s) may be utilized. The non-signal medium may be a storage medium. A storage medium may be, for example, an electronic, magnetic, optical, electromagnetic, infrared, or semiconductor system, apparatus, or device, or any suitable combination of the foregoing. More specific examples of a storage medium would include the following: a portable computer diskette, a hard disk, a random access memory (RAM), a dynamic random access memory (DRAM), a read-only memory (ROM), an erasable programmable read-only memory (EPROM or Flash memory), a portable compact disc read-only memory (CD-ROM), an optical storage device, a magnetic storage device, or any suitable combination of the foregoing.

Program code for carrying out operations may be written in any combination of one or more programming languages. The program code may execute entirely on a single device, partly on a single device, as a stand-alone software package, partly on single device and partly on another device, or entirely on the other device. In some cases, the devices may be connected through any type of network, including a local area network (LAN) or a wide area network (WAN), or the connection may be made through other devices (for example, through the Internet using an Internet Service Provider) or through a hard wire connection, such as over a USB connection. For example, a server having a first processor, a network interface, and a storage device for storing code may store the program code for carrying out the operations and provide this code through its network interface via a network to a second device having a second processor for execution of the code on the second device.

Aspects are described herein with reference to the Figures, which illustrate example methods, devices and program products according to various example embodiments. These program instructions may be provided to a processor of a general purpose computer, special purpose computer, or other programmable data processing device or information handling device to produce a machine, such that the instructions, which execute via a processor of the device implement the functions/acts specified. The program instructions may also be stored in a device readable medium that can direct a device to function in a particular manner, such that the instructions stored in the device readable medium produce an article of manufacture including instructions which implement the function/act specified. The program instructions may also be loaded onto a device to cause a series of operational steps to be performed on the device to produce a device implemented process such that the instructions which execute on the device provide processes for implementing the functions/acts specified.

The units/modules/applications herein may include any processor-based or microprocessor-based system including systems using microcontrollers, reduced instruction set computers (RISC), application specific integrated circuits (ASICs), field-programmable gate arrays (FPGAs), logic circuits, and any other circuit or processor capable of executing the functions described herein. Additionally or alternatively, the modules/controllers herein may represent circuit modules that may be implemented as hardware with associated instructions (for example, software stored on a tangible and non-transitory computer readable storage medium, such as a computer hard drive, ROM, RAM, or the like) that perform the operations described herein. The above examples are exemplary only, and are thus not intended to limit in any way the definition and/or meaning of the term “controller.” The units/modules/applications herein may execute a set of instructions that are stored in one or more storage elements, in order to process data. The storage elements may also store data or other information as desired or needed. The storage element may be in the form of an information source or a physical memory element within the modules/controllers herein. The set of instructions may include various commands that instruct the modules/applications herein to perform specific operations such as the methods and processes of the various embodiments of the subject matter described herein. The set of instructions may be in the form of a software program. The software may be in various forms such as system software or application software. Further, the software may be in the form of a collection of separate programs or modules, a program module within a larger program or a portion of a program module. The software also may include modular programming in the form of object-oriented programming. The processing of input data by the processing machine may be in response to user commands, or in response to results of previous processing, or in response to a request made by another processing machine.

It is to be understood that the subject matter described herein is not limited in its application to the details of construction and the arrangement of components set forth in the description herein or illustrated in the drawings hereof. The subject matter described herein is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

It is to be understood that the above description is intended to be illustrative, and not restrictive. For example, the above-described embodiments (and/or aspects thereof) may be used in combination with each other. In addition, many modifications may be made to adapt a particular situation or material to the teachings herein without departing from its scope. While the dimensions, types of materials and coatings described herein are intended to define various parameters, they are by no means limiting and are illustrative in nature. Many other embodiments will be apparent to those of skill in the art upon reviewing the above description. The scope of the embodiments should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects or order of execution on their acts.

What is claimed is:

1. An ultrasound system, comprising:
a probe that is operable to transmit ultrasound signals and receive echo ultrasound signals from a region of interest (ROI); and
processing circuitry that is operable to:
perform a first beamforming operation on at least a portion of the echo ultrasound signals to generate a first ultrasound dataset, corresponding to at least a portion of a first ultrasound image, the first beamforming operation performing beamforming for a subregion of the ROI utilizing an initial time delay as a beamforming parameter;
apply a deep learning network (DLN) model to a local region of the first ultrasound dataset to identify at least one of a tissue type or density characteristic associated with the local region;
adjust the beamforming parameter to use a density adjusted (DA) time delay based on the at least one of a tissue type or density characteristic of the local region, to form a density adjusted beamforming (DAB) parameter; and
perform a second beamforming operation on at least a portion of the echo ultrasound signals, based on the DA time delay for the DAB parameter, to generate a second ultrasound dataset.

2. The system of claim 1, wherein the processing circuitry is further operable to segment the first ultrasound dataset into multiple local regions and, for at least a portion of the local regions, repeat the first and second beamforming operations, applying the DLN model and adjusting the DAB parameter.

3. The system of claim 1, wherein the TDB parameter and DAB parameter include different first and second sets of time delays that are utilized during the first and second beamforming, respectively, in connection with a common segment of the ROI.

4. The system of claim 1, wherein the first and second beamforming operations are performed on a common portion of the echo ultrasound signals.

5. The system of claim 1, wherein the probe is operable to perform first and second scans of the ROI, during which first and second sets of the echo ultrasound signals are received, the first scan performed before the first beamforming operation, the second scan performed after the first beamforming operation and before the second beamforming operation.

6. The system of claim 1, wherein the DLN model classifies the local regions to correspond to one of at least two different types of tissue, the types of tissue including at least two of air, lung, fat, water, brain, kidney, liver, myocardium, or bone.

7. The system of claim 1, wherein the TDB parameter includes a first time delay value associated with a reference density, the processing circuitry operable to adjust the TDB parameter to form the DAB parameter by changing the first time delay value to a second time delay value associated with a predicted density corresponding to the at least one of a tissue type or density characteristics identified by the DLN model.

8. The system of claim 7, wherein the second time delay value is determined based on a propagation time from an array element of the probe to a focal point in the ROI utilizing a predicted speed of sound that is determined based on the at least one of a tissue type or density characteristics identified by the DLN model.

9. The system of claim 2, wherein the second ultrasound dataset is based on second ultrasound signals that are received after adjusting the beamforming parameter, the second ultrasound dataset corresponding to a second ultrasound image.

10. The system of claim 1, wherein the processing circuitry is operable to segment the first ultrasound dataset into a two-dimensional array of the local regions, wherein each of the local regions corresponds to a different portion of the ultrasound image.

11. A computer implemented method, comprising:
utilizing an ultrasound probe to transmit ultrasound signals and receive echo ultrasound signals from a region of interest;
under control of processing circuitry:
performing first beamforming on at least a portion of the echo ultrasound signals to generate a first ultrasound dataset, corresponding to a first ultrasound image, based on a time delay beamforming (TDB) parameter;
applying a deep learning network (DLN) model to the local regions to identify at least one of a tissue type or density characteristics associated with corresponding portions of the ROI in the associated local regions;
adjusting the TDB parameter, based on the at least one of a tissue type or density characteristics of the corresponding local regions, to form a density adjusted beamforming (DAB) parameter; and
performing second beamforming on at least a portion of the echo ultrasound signals, based on the DAB parameter, to generate a second ultrasound dataset.

12. The method of claim 11, wherein the first and second beamforming are performed on a common portion of the echo ultrasound signals.

13. The method of claim 11, wherein the probe is operable to perform first and second scans of the ROI, during which first and second sets of the echo ultrasound signals are received, the first scan performed before the first beamforming operation, the second scan performed after the first beamforming operation and before the second beamforming operation.

14. The method of claim 11, wherein the DLN model classifies the local regions to correspond to one of at least two different types of tissue, the types of tissue including at least two of air, lung, fat, water, brain, kidney, liver, myocardium, or bone.

15. The method of claim 11, wherein the TDB parameter includes a first time delay value associated with a reference density, the processing circuitry operable to adjust the TDB parameter to form the DAB parameter by changing the first time delay value to a second time delay value associated with a predicted density corresponding to the at least one of a tissue type or density characteristics identified by the DLN model.

16. The method of claim 11, wherein the second time delay value is determined based on a propagation time from an array element of the probe to a focal point in the ROI utilizing a predicted speed of sound that is determined based on the at least one of a tissue type or density characteristics identified by the DLN model.

17. The method of claim 11, wherein the second ultrasound dataset is based on second ultrasound signals that are received after adjusting the DAB parameter, the second ultrasound dataset corresponding to a second ultrasound image.

18. A system comprising:
memory to store program instructions; and
one or more processors that, when executing the program instructions, are configured to:
obtain a collection of reference images for a patient population, the reference images representing ultrasound images that are obtained from a patient population having different types of tissue for one or more anatomical regions; and
analyze the collection of reference images utilizing a deep learning network (DLN) to define a DLN model that is configured to identify different types of anatomical regions and different density properties within the corresponding anatomical regions.

19. The system of claim 18, wherein the one or more processors are configured to analyze the collection of reference images by performing one or more convolutions and up sampling operations to generate a feature map.

20. The system of claim 18, wherein the one or more processors are configured to train the DLN model by minimizing a sigmoid cross loss objective.
